(19) United States
(12) Patent Application Publication
(10) Pub. No.: US 2004/0001886 A1
(43) Pub. Date: Jan. 1, 2004

(54) Stabilized pharmaceutical formulations containing amlodipine maleate

(75) Inventors: Dinesh Dayaramji Chakole, Hyderabad (IN); Pallempalli Venkata Siva Reddy, Hyderabad (IN); Billa Praveen Reddy, Hyderabad (IN); Vipin Tatyasaheb Dhanorkar, Hyderabad (IN); Mailatur Sivaraman Mohan, Hyderabad (IN)

Correspondence Address: LADAS & PARRY, 26 WEST 61ST STREET, NEW YORK, NY, 10023, US

(73) Assignee: DR. REDDY'S LABORATORIES LIMITED

(21) Appl. No.: 10/417,810

(22) Filed: Apr. 17, 2003

Related U.S. Application Data
(63) Continuation of application No. 10/244,049, filed on Sep. 13, 2002.

(30) Foreign Application Priority Data
Oct. 17, 2001 (IN) 852/MAS/2001
Jul. 18, 2002 (WO) PCT/US02/22908

(51) Int. Cl. A61K 31/724; A61K 31/455; A61K 9/20; A61K 9/36

(52) U.S. Cl. 424/465; 514/58; 514/60; 514/355; 424/480

(57) ABSTRACT

The present invention relates to the stable solid orally administrable pharmaceutical formulation of Amlodipine Maleate. The invention also describes the process of producing such stable formulations and more specifically a direct compression method of producing tablet formulations. The tablet formulation of Amlodipine Maleate thus prepared is bioequivalent to the tablets containing Amlodipine Besylate salt commercially available with the brand name of Norvasc. The formulation also avoids the common problem of sticking observed during manufacturing.

Stable formulations comprising Amlodipine Maleate are disclosed.

Amlodipine belongs to dihydropyridine group of compounds having chemical name (±)-2-[(2-Aminoethoxy) methyl]-4-(2-chlorophenyl)-3-ethoxycarbonyl-5-methoxycarbonyl-6-methyl-1,4-dihydropyridine and molecular formula C₂₀—H₂₅—Cl—N₂—O₅. It exists in various salt forms amongst which is Amlodipine Besylate. Amlodipine Besylate is commercially available as Norvasc in the form of oral tablets by Pfizer in 2.5 mg, 5 mg and 10 mg base preparations. Therapeutically Amlodipine belongs to the class of antianginals and antihypertensives. The main mechanism of action of Amlodipine is the inhibition of calcium channels. It is also available in combination with diuretics and angiotensin converting enzyme inhibitors.

Amlodipine is well absorbed through gut. Oral bioavailability of Amlodipine in man was found to be 64%. Plasma protein binding for Amlodipine is high in man corresponding to 97%.

Use of Amlodipine in the therapy of cardiovascular disorders is known. Patent specification AU1354000 discloses a method for treating hypertension, angina and other disorders using optically pure (−) Amlodipine. U.S. Pat. No. 6,080,761 discloses the inhibition of smooth muscle migration by (R) Amlodipine. Flynn J T et al. describes the Treatment of hypertensive children with Amlodipine in Am. J. Hypertens. (AJHYE6, 08957061); 2000; Vol. 13 (10); pp. 1061-1066. Marche P discloses Amlodipine and the mechanisms of vascular hypertrophy in Drugs (DRUGAY, 00126667); 2000; Vol.59 (Spec. Issue 2); pp.1-7. Burges R A explains the Pharmacologic profile of Amlodipine Am. J. Cardiol. (AJCDAG, 00029149); 1989; Vol.64 (17); pp.10I-20I.

The process of preparation of various salts of Amlodipine is also known in the prior art. Patent specification KR217240 discloses a method for the preparation of Amlodipine Besylate. Teachings of U.S. Pat. No. 5,438,145 are devoted to a process for the preparation of Amlodipine benzenesulphonate.

U.S. Pat. No. 4,572,909, discloses several different pharmaceutically acceptable acid addition salts of Amlodipine. In particular, the pharmaceutically acceptable acid addition salts are said to be those formed from acids which form non toxic acid anions such as hydrochloride, hydrobromide, sulphate, phosphate, acetate, maleate, lactate, tartrate, citrate, gluconate etc. of these salts maleate is disclosed as being particularly preferred.

U.S. Pat. No. 4,879,303 teaches that besylate salt of Amlodipine has a number of advantages over the known salt of Amlodipine, and has a unique combination of good formulation properties that make it particularly suitable for preparation of pharmaceutical formulations of Amlodipine. Besylate salt of Amlodipine is particularly disclosed in U.S. Pat. No. 4,879,303 as having good processability, solubility, stability and nonhygroscopicity. The teaching of U.S. Pat. No. 4,879,303 suggests that maleate salts of Amlodipine are more prone to degradation especially in solution form. The question of stability of Amlodipine has long been a part of the discussion in the prior art.

U.S. Pat. No. 4,879,303 describes besylate salt of Amlodipine as possessing superior antiadhesion properties as compared to the maleate salt. It has been observed that Amlodipine Maleate is very sensitive to moisture and interacts with certain formulation excipients to undergo degradation. It has been observed that Amlodipine Maleate along with having stability constraints poses a problem of sticking during manufacturing of tablets. Sticking causes drug to accumulate on the punch surfaces resulting in pitted tablets which is unacceptable. The sticking of the tablets in this way results in high ejection forces when removing the tablets from the machine.

US Publication 2002/0086888 of Jul. 4, 2002 and WO 02/053542 describe a number of processes for preparing amlodipine maleate.

WO 02/05134 discloses formulations containing amlodipine maleate which include calcium hydrogen phosphate anhydrous, microcrystalline cellulose, sodium starch glycollate and magnesium stearate. An advantage of the formulations of this invention is that avoiding the use of dibasic calcium phosphate results in more stable formulations.

The present invention is directed towards preparation of solid orally administrable dosage form of Amlodipine Maleate which overcomes the aforementioned problems. The solid oral formulation of the present invention involves the use of pharmaceutically and/or physiological acceptable diluents, disintegrants, carriers, and lubricants and amlodipine maleate. The Amlodipine Maleate used in the present invention may be prepared as per the process disclosed in U.S. Pat. No. 4,572,909 or by any other process.

The formulations of this invention contain about 0.25 to about 7% by weight of amlodipine maleate; about 50 to about 97% by weight of a diluent; about 0.5 to about 10% by weight of a disintegrant; about 0.25 to about 2% by weight of a lubricant and about 0.1 to about 2.5% by weight of a glidant.

Suitable diluents include but are not limited to polyols selected from mannitol, sorbitol, and xylitol; sugars selected from sucrose, lactose, and dextrose; cellulose derivatives selected from microcrystalline cellulose, hydroxypropyl cellulose, and powdered cellulose; and saccharides selected from dextrates, maltodextrans, starches, pregelatinized starch, cyclodextrin and the like.

The disintegrants that may be used include but are not limited to one or more clays selected from bentonite, vegum and the like; one or more gums selected from sodium alginate, xanthan gum, veegam, guar gum and the like; one or more polymers selected from sodium starch glycolate, crosscarmellose sodium, crosslinked polyvinyl-pyrrolidone, crospovidone, and the like. The polymer as used herein refers to polymeric disintegrants typically used in the pharmaceutical industry.

The lubricants that may be used include but are not limited to talc, stearic acid, magnesium stearate, glyceryl behenate, behenic acid, hydrogenated vegetable oils, calcium stearate, mineral oil, polyethylene glycols, sodium lauryl sulphate, and the like.

The glidants that may be used include but are not limited to talc, colloidal silicon dioxide, and the like.

In a preferred embodiment of the invention the composition comprises about 0.25 to about 7% by weight of amlodipine maleate, about 50 to about 97% by weight of microcrystalline cellulose, about 0.5 to about 10% by weight of sodium starch glycolate, about 0.25 to about 2% by weight of magnesium stearate; and about 0.1% to about 2.5% by weight of colloidal silicon dioxide.

In a more preferred embodiment of the invention the composition comprises about 1 to about 5% by weight of amlodipine maleate, about 75 to about 95% by weight of microcrystalline cellulose, about 0.5 to about 5% by weight of sodium starch glycolate, about 0.5 to about 2% by weight of magnesium stearate; and about 0.2 to about 2% by weight of colloidal silicon dioxide.

A still more preferred formulation of the invention is a composition that comprises about 3 to about 5% by weight of amlodipine maleate, about 90 to about 95% by weight of microcrystalline cellulose, about 1 to about 2% by weight of sodium starch glycolate, about 1 to about 2% by weight of magnesium stearate; and about 0.2 to about 2% by weight of colloidal silicon dioxide.

In another embodiment of the invention the composition comprises about 0.25% to about 7% by weight of amlodipine maleate, about 50% to about 97% by weight of mannitol, about 0.5% to about 10% by weight of croscarmellose sodium, about 0.25% to about 2% by weight of magnesium stearate, and about 0.25% to about 2.5% by weight of colloidal silicon dioxide.

In another embodiment of the invention the composition comprises about 0.25% to about 7% by weight of amlodipine maleate, about 50% to about 97% by weight of pregelatinized starch, about 0.5% to about 10% by weight of crospovidone, about 0.25% to about 2% by weight of magnesium stearate and about 0.25 to about 2.5 by weight of colloidal silicon dioxide.

Preferably the direct compression method is used to produce the stable Amlodipine Maleate tablets. In one embodiment of the present invention the problem of sticking commonly observed with Amlodipine Maleate formulations has been reduced or eliminated and stable formulations of Amlodipine Maleate have been produced without using any stabilizing agent. The prepared Amlodipine Maleate tablets were also found to be bioequivalent with the Amlodipine Besylate tablet commercially available as Norvasc in the US market.

As used herein “bioequivalent” refers to pharmaceutical equivalent or pharmaceutical alternative products that display comparable bioavailability when studied under similar experimental conditions. A test drug and a reference listed drug shall be considered bioequivalent if:

The rate and extent of absorption of the test drug do not show a significant difference from the rate and extent of absorption of the reference drug when administered at the same molar dose of the therapeutic ingredient under similar experimental conditions in either a single dose or multiple doses; or

The extent of absorption of the test drug does not show a significant difference from the extent of absorption of the reference drug when administered at the same molar dose of the therapeutic ingredient under similar experimental conditions in either a single dose or multiple doses and the difference from the reference drug in the rate of absorption of the drug is intentional, (is reflected in its proposed labeling), is not essential to the attainment of effective body drug concentrations (on chronic use), and is considered medically insignificant for the drug.

Other definitions and tests of bioequivalence can be used if desired, to determine bioequivalence between a test drug and a reference drug.

The term “bioavailability” refers to the rate and extent to which the active ingredient or active moiety is absorbed from a drug product and becomes available at the site of action.

As used herein the term “stable solid orally administrable pharmaceutical formulations” refer to the oral solid dosage form of Amlodipine Maleate wherein the impurity 2 (3,5-dimethyl-2-[(2-aminoethoxy)methyl]-4-2-chlorophenyl)-6-methyl-1,4-dihydropyridine-3,5-carboxylate and impurity 6 (3-ethyl-5-methyl-2-[{2-(N-succinyl)amino)ethoxy}methyl]-4-{2-chlorophenyl}-6-methyl-1,4-dihydropyridine-3,5 -dicarboxylate) does not exceed more than 0.5% by weight and 0.8% by weight respectively upon exposure of temperature at conditions of 40° C./75% relative humidity (RH), 25° C./60% RH or 60° C. for six month period. The total impurity including impurity 2 and 6 should not exceed more than 2% during the shelf life of the product. The meaning of stable according to this invention should not be construed to limit to the temperature exposures period/conditions and impurity 2 or 6 as described in this specification. Stable also refers to the oral formulations of Amlodipine Maleate which satisfy the criteria of ICH guidelines for stability and FDA requirement for regulatory submissions.

The oral formulations of this invention may be in the form of tablets, caplets and capsules. Other solid oral formulation are also within the scope of the invention.

The antihypertensive or antianginal alleviating effective amount of Amlodipine Maleate according to this invention comprises from 2.0 mg to 10 mg dosage of Amlodipine Maleate.

The following examples illustrate but do not limit the scope of the invention.

### EXAMPLE 1

Wet Granulation Method

The ingredients used in the preparation of Amlodipine Maleate tablet by wet granulation method are given below along with the method for the preparation of said tablets.

| | Quantity¹ | | Quantity² In |
|---|---|---|---|
| Ingredients | mg/tablet | | weight % |
| Amlodipine Maleate | 12.84 | | 0.25 to 7 |
| Microcrystalline | 229.16 | | 40 to 80 |
| cellulose | | | |
| Dibasic calcium | 126.00 | | 20 to 80 |
| phosphate | | | |
| Polyvinylpyrrolidone | 12 | | 1.5 to 5 |
| Sodium starch | 16 | | 0.5 to 6 |
| glycolate | | | |
| Magnesium stearate | 4 | | 0.25 to 2 |
| Tablet weight | 400 | mg | 100% |

¹actual amounts used
²range of weight % that can be used in a formulation.

Procedure

Amlodipine Maleate, microcrystalline cellulose and dibasic calcium phosphate are passed through mesh # 40 screen and blended together. Polyvinyl-pyrrolidone is dissolved in water and this solution is used as granulating solution to prepare granules of the above blend. The granules after drying were blended with magnesium stearate previously sifted through mesh # 40. The final blend thus obtained was compressed into tablets.

Stability Profile

The initial and one month stability profile for Amlodipine Maleate tablets prepared by above wet granulation method is shown below.

The stability studies are carried out at 40° C. and 75% relative humidity conditions.

| | Stability data | | |
|---|---|---|---|
| Time | % weight | % weight | % weight Total |
| Period | Impurity 2 | Impurity 6 | Impurity |
| Initial | 0.08 | 0.8 | 1.0 |
| 1 Month | 0.2 | 1.8 | 2.3 |

It was observed that the granules obtained by the wet granulation method of Example 1 were very fragile and colour of granules changed to pale yellow on standing. It was observed that Amlodipine Maleate shows degradation and yielded impurities as degradation products referred to as impurity 2 and impurity 6. It is noted that the above method includes the use of water to prepare the binder solution of polyvinylpyrrolidone. Since the active ingredient used in the above formulation, Amlodipine Maleate, is susceptible to hydrolytic degradation, as supported by prior art, it may be presumed that the impurities generated were because of the presence of water or moisture in the formulation process.

In order to confirm the above observation another tablet formulation of Amlodipine Maleate was tried which minimizes the use of water in the manufacturing steps. In this formulation isopropyl alcohol is used as granulating solution, which is presented below as Example 2.

### EXAMPLE 2

Solvent Granulation with Isopropyl Alcohol

The ingredients used in this process are listed below.

| | Quantity¹ | | Quantity² in |
|---|---|---|---|
| Ingredients | mg/tablet | | weight % |
| Amlodipine Maleate | 12.84 | | 0.25 to 7 |
| Microcrystalline cellulose | 244.76 | | 40 to 80 |
| Dibasic calcium phosphate | 126.00 | | 20 to 80 |
| Sodium starch glycolate | 8 | | 0.5 to 16 |
| Polyoxyl-40-hydrogenated castor oil | 4.4 | | 0.25 to 10 |
| Magnesium stearate | 4 | | 0.25 to 20 |
| Tablet weight | 400 | mg | 100% |

¹actual amounts used
²range of weight % that can be used in a formulation.

Procedure

Polyoxyl-40-hydrogenated castor oil was dissolved in sufficient quantity of water. Care was taken to keep the water used to minimum i.e. sufficient enough to facilitate dissolution of Polyoxyl-40-hydrogenated castor oil. The above Polyoxyl-40-hydrogenated castor oil solution was added to isopropyl alcohol taken in a quantity sufficient to dissolve polyoxyl-40-hydrogentated castor oil and dibasic calcium phosphate mixture used in the weight ratio of 1:1.

Amlodipine Maleate was then added to above mixture for solubilization. This solution was then mixed with previously sifted microcrystalline cellulose (# 40) to obtain a weight mass. The wet mass thus obtained was dried at 50° C. for 30 minutes. Dried mass was passed through # 40 screen and mixed with sodium starch glycolate and magnesium stearate (previously sifted through # 40) with proper blending in double cone blender for about 10 minutes. The final blend thus obtained was compressed into tablets.

Stability Profile

The initial and one month stability profile for tablets prepared with above isopropyl alcohol solvent granulation process is shown below.

The stability studies are carried out at 40° C. and 75% relative humidity conditions.

| | Stability data | | |
|---|---|---|---|
| Time | % Weight | % Weight | % Weight Total |
| Period | Impurity 2 | Impurity 6 | Impurity |
| Initial | 0.02 | 0.05 | 0.2 |
| 1 Month | 0.8 | 0.7 | 2.0 |

It is evident from the impurity profile that even when use of water was restricted to minimum in the above formulation, it showed an impurity profile similar to that observed in case of wet granulation process described in Example 1. This was an indication that Amlodipine Maleate is very sensitive to the presence of moisture in the formulation which is also supported by the prior art.

It was mentioned in the prior art that salts of Amlodipine undergo degradation especially in solutions. A pH of solution directly affects the stability of Amlodipine salts in formulations. In order to find out the effect of pH on stability of Amlodipine Maleate formulations a basic formulation was prepared with meglumine as basifying agent.

### EXAMPLE 3

Amlodipine Maleate Tablet Containing Meglumine

The ingredients used in the formulation containing meglumine along with Amlodipine Maleate are listed below.

| | Quantity¹ | Quantity² |
|---|---|---|
| Ingredient | in mg/Tablet | in weight % |
| Amlodipine Maleate | 12.84 | 0.25 to 7 |
| Microcrystalline cellulose | 293.46 | 50 to 97 |
| Sodium starch glycolate | 4.5 | 0.5 to 5 |
| Magnesium stearate | 4.5 | 0.25 to 5 |
| Colloidal silicon dioxide | 1.5 | 0.1 to 2.5 |
| Meglumine | 3.2 | 0.25 to 3 |
| Tablet weight | 320.0 | 100% |

¹actual amounts used
²range of weight % that can be used in a formulation.

Procedure

Amlodipine Maleate, microcrystalline cellulose (18%), sodium starch glycolate, meglumine and colloidal silicon dioxide (87% of the total) were sifted through # 40 screen. The remaining quantity of microcrystalline cellulose was sifted through # 40 screen along with the above-sifted material and blended in double cone blender. Remaining quantity of colloidal silicon dioxide and magnesium stearate were mixed in above blend for the purpose of lubrication. The resulting blend is compressed in to tablets or can be alternatively filled into capsule.

The pH of the above tablet formulation was found to be 6.95 in contrast to the pH of the Amlodipine Besylate tablet, Norvasc, which was 7.79.

Stability Profile

Stability data (Direct exposure study)

| Time and condition of | Weight % | Weight % | Weight % Total |
|---|---|---|---|
| exposure | Impurity 2 | Impurity 6 | Impurity |
| Initial | 0.17 | 0.09 | 0.35 |
| 40° C./75% RH-15 Days | 0.06 | 2.83 | 3.1 |
| 40° C./75% RH-30 Days | 0.09 | 8.86 | 9.7 |
| 60° C.-15 Days | 0.06 | 0.47 | 0.7 |
| 60° C.-30 Days | 0.09 | 1.32 | 1.67 |

It was very important observation that even under basic environment Amlodipine showed degradation and the level of impurity 6 still increased.

Impurity Identification

In an effort to explore further stable formulations of Amlodipine Maleate, it was necessary to study the chemical nature and identify the impurity profile of Amlodipine Maleate observed during processing in Examples 1 and 2. These were identified as impurities 2 and 6 as described below.

Procedure

The procedure to identity the related substance is official in US Pharmacopoeia. The various parameters used for identification of related substance are given below.

HPLC method:

Liquid chromatograph equipped with 237 nm UV detector.

Column: 250 mm×4.00 mm column that contains 5 microns packing of octyl silane chemically bonded to porous silica or ceramic micro particles.

Column temperature: Ambient, Flow rate: 1.0 ml per minutes.

Details of the impurities observed with processing of Amlodipine Maleate Impurity 2: 3,5-Dimethyl-2 [(2-aminoethyoxy)methyl]-4-2-chlorophenyl)-6-methyl-1,4-dihydropyridine-3,5-dicarboxylate(dimethyl Amlodipine)

Relative retention time: about 0.71%.

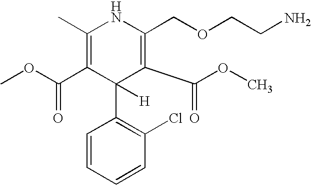

This impurity is not mentioned in European Pharmacopoeia but this can be identified by European Pharmacopoeia HPLC method. This impurity is known as Dimethoxy Carbonyl Amlodipine impurity. This impurity is formed due to presence of methyl, 4-chloro acetoacetate in the raw material ethyl, 4-chloro acetoacetate. This impurity is controlled by the quality of raw material ethyl, 4-chloro acetoacetate. The limit for this impurity is fixed as not more than 0.2% by weight.

Impurity 6

3-ethyl-5-methyl-2-[{2-(N-succinyl)amino)ethoxy}methyl]-4-{2-chlorophenyl}-6-methyl-1,4-dihydropyridine-3,5-dicarboxylate

Relative retention time: about 0.91%.

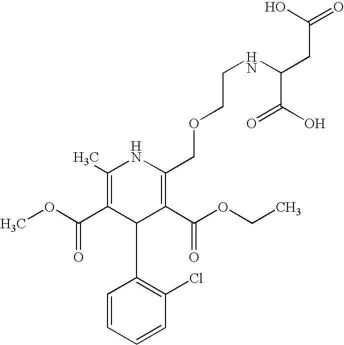

This impurity is not mentioned in European Pharmacopoeia but this can be identified by European Pharmacopoeia HPLC method and elutes at about 0.9% relative retention time. This impurity is formed by reacting the amino group of Amlodipine base at the alpha carbon of Maleic acid. Higher temperatures and basic conditions are favorable to form this impurity. It is found that this impurity is enhanced during the formulation of Amlodipine Maleate. This impurity can be controlled by the reaction condition. The limit for this impurity has not been fixed.

In case of conditions of 40° C./75% RH (relative humidity) the level of impurity 6 is increased as compared to that observed at 60° C. The level of impurity 2 generally was unaffected in almost all the formulations.

The Amlodipine Maleate tablets prepared in Examples 1, 2 and 3 had a problem of sticking which ultimately lead to the problem of content uniformity in the final dosage form. This problem of sticking along with the unsolved problem of stability required the need of further formulation trials.

### EXAMPLE 4

Elimination of Sticking During Formulation of Amlodipine Tablets

To eliminate sticking problem commonly observed during formulation trials of Amlodipine Maleate different experiments were conducted as illustrated in following table.

| | | | | | | Quantity² |
|---|---|---|---|---|---|---|
| | Quantity¹ mg/tablet | | | | | in weight |
| Ingredients | 1 | 2 | 3 | 4 | 5 | % |
| Amlodipine Maleate | 12.84 | 12.84 | 12.84 | 12.84 | 12.84 | 0.25 to 7 |
| Microcrystalline cellulose | — | 139.08 | 126.58 | 183.66 | 296.66 | 50 to 97 |
| Lactose monohydrate | 353.16 | 139.08 | 126.58 | — | — | 50 to 95 |
| Pregelatinized starch | 30.0 | — | — | — | — | 2 to 15 |
| Calcium sulphate | — | — | 25 | — | — | 2 to 10 |
| Dibasic calcium | — | — | — | 94.5 | — | 20 to 80 |
| phosphate | | | | | | |
| Sodium starch glycolate | 8.0 | 6.0 | 6.0 | 6.0 | 4.5 | 0.5 to 10 |
| Colloidal silicon dioxide | — | — | — | — | 1.5 | 0.1 to 2.5 |
| Magnesium stearate | 4.0 | 3.0 | 3.0 | 3.0 | 4.5 | 0.2 to 2 |
| Total weight (mg) | 408 | 300 | 300 | 300 | 320 | 100% |
| Sticking | Yes | Yes | Yes | Yes | No | — |

¹actual amounts used
²range of weight % that can be used in a formulation.

With the extensive further trials in order to improve the formulation towards achieving considerable stability it was surprisingly noted that avoiding dicalcium phosphate yielded more Amlodipine Maleate stable formulations. In contrast use of dibasic calcium phosphate with Amlodipine Besylate yielded stable formulations. The stabilized formulation of Amlodipine after extensive trials that also avoided the sticking problem commonly observed, has been optimized and presented below as a prototype example. Obvious variations in the examples of Amlodipine Maleate formulations are contemplated to be within the scope of the present invention.

### EXAMPLE 5

Direct Compression Method for Producing Amlodipine Maleate Tablets

The following are stable formulations containing Amlodipine Maleate as 2.5, 5.0 and 10 mg tablet preparations.

| | Quantity¹ mg/tablet | | | Quantity² |
|---|---|---|---|---|
| Ingredients | 2.5 mg | 5 mg | 10 mg | in weight % |
| Amlodipine Maleate | 3.21 | 6.42 | 12.84 | 0.25 to 7 |
| Microcrystalline cellulose | 74.165 | 148.33 | 296.66 | 50 to 97 |
| Sodium starch glycolate | 1.125 | 2.25 | 4.5 | 0.5 to 10 |
| Magnesium stearate | 1.125 | 2.25 | 4.5 | 0.25 to 2 |
| Colloidal silicon dioxide | 0.375 | 0.75 | 1.50 | 0.1 to 2.5 |
| Tablet weight (mg) | 80 | 160 | 320 | 100 |

¹actual amounts used
²range of weight % that can be used in a formulation.

Procedure

All the above ingredients are sifted through # 40 screen. Above ingredients except magnesium stearate and colloidal silicon dioxide are blended in double cone blender for about 30 minutes. The final moisture content of the blend was maintained in the range of about 2.5 to about 4.5% by weight. Magnesium stearate and colloidal silicon is added to the above blend and blended for about 5 minutes. The resulting final blend is then compressed into tablets or alternatively filled into capsules.

All the operations for the manufacturing of formulation referred above as stable formulation, which are confirmed with reference to following stability and impurity profile, are carried out under conditions of relative humidity of 45% and temperature of 25° C.

Stability Profile

3 Month

| | | | |
|---|---|---|---|
| Impurities* | Initial | 40° C./75% RH | 25° C./60% RH |

Amlodipine Maleate 2.5 mg tablet

| | | | |
|---|---|---|---|
| Impurity 2 | 0.1 | 0.15 | 0.17 |
| Impurity 6 | 0.1 | 0.7 | 0.06 |
| Total | 0.3 | 1.0 | 0.3 |

*Impurities are expressed in percentages by weight.

Amlodipine Maleate 5 mg tablet

| | | | |
|---|---|---|---|
| Impurity 2 | 0.2 | 0.18 | 0.17 |
| Impurity 6 | 0.09 | 0.16 | 0.16 |
| Total | 0.3 | 0.4 | 0.4 |

*Impurities are expressed in percentages by weight.

Amlodipine Maleate 10 mg tablet

| | | | |
|---|---|---|---|
| Impurity 2 | 0.1 | 0.18 | 0.17 |
| Impurity 6 | 0.1 | 0.24 | 0.07 |
| Total | 0.3 | 0.5 | 0.3 |

*Impurities are expressed in percentages

It should be noted that the level of impurity 6 in Example 5, which was considered as the major impurity, was minimized drastically compared to that observed during earlier examples. In most of the cases impurity 2 remained more or less unaffected.

### EXAMPLE 6

The stability of above Amlodipine Maleate tablet formulation was compared with Amlodipine Besylate tablets commercially available as Norvasc 6.

Norvasc Composition

The commercially available NORVASC comprises Dibasic calcium phosphate, Microcrystalline cellulose, Sodium starch glycolate, Magnesium stearate and Amlodipine Besylate.

Norvasc 10 mg Tablet

| | | 3 Month |
|---|---|---|
| Impurities* | Initial | 40° C./75% RH |
| Impurity 2 | 0.00 | 0.29 |
| Impurity 4 | 0.01 | 0.04 |
| Total | 0.1 | 0.5 |

*Impurities are expressed in percentages by weight.

It should be noted that the impurity referred to above as impurity 6 is not observed with Norvasc 10 mg tablet formulations. The impurity 4 is characterized as 3,5-dimethyl-(4 RS)-4-(2-chlorophenyl)-2-[(2-aminoethoxy) methyl]-6-methyl-1,4-dihydropyridine-3,5-dicarboxylate (diethoxy Amlodipine) with the RRT (relative retention time) of 1.60.

In order to protect the Amlodipine from hydrolytic degradation, the tablets are coated with solution of coating agent commonly employed in pharmaceutical formulations. These coated tablet formulations were then compared with uncoated tablet formulations of Amlodipine Besylate with respect to stability and impurity profile. The data is presented below.

### EXAMPLE 7

Coated Tablets containing Amlodipine Maleate 10 mg

| | | Quantity¹ | Quantity² |
|---|---|---|---|
| S.N. | INGREDIENTS | mg/Tablet | in weight % |
| 1 | Amlodipine Maleate | 12.84 | 0.25 to 7 |
| 2 | Microcrystalline cellulose | 296.66 | 50 to 97 |
| 3 | Sodium starch glycolate | 4.5 | 0.5 to 10 |
| 4 | Magnesium stearate | 4.5 | 0.25 to 2 |
| 5 | Colloidal silicon dioxide | 1.5 | 0.25 to 2.5 |
| | Tablet weight (core) | 320.0 | |

Coating

| | | | |
|---|---|---|---|
| 6 | Opadry pink OY 54937 | 12.8 | 2.5 to 5 |
| | Tablet weight (coated) | 332.8 | 100% |

¹actual amounts used
²range of weight % that can be used in a formulation.
*Opadry is a mixture of hydroxypropylmethylcellulose, titanium dioxide, polyethylene glycol 400 and Synthetic iron oxide.

All the above ingredients are sifted through # 40 screen. Above ingredients except magnesium stearate and colloidal silicon dioxide are blended in Double cone blender for about 30 minutes. Magnesium stearate and colloidal silicon is added to the above blend and the resulting final blend is then compressed into tablets.

Stability Profile

Stability data (Direct exposure study)

| Condition of | | Weight % | Weight % | Weight % |
|---|---|---|---|---|
| exposure | Time | Impurity 2 | Impurity 6 | Total Impurity |
| | Initial | 0.1 | 0.1 | 0.3 |
| 40° C./75% RH | 15 Days | 0.1 | 0.9 | 1.2 |
| | 30 Days | 0.15 | 1.69 | 2.3 |
| 60° C. | 15 Days | 0.1 | 0.77 | 1.3 |
| | 30 Days | 0.14 | 1.2 | 1.9 |

### EXAMPLE 8

Coated Tablets containing Amlodipine Maleate 10 mg

| | | Quantity¹ | Quantity² in |
|---|---|---|---|
| S.N. | INGREDIENTS | mg/Tablet | weight % |
| 1 | Amlodipine Maleate | 12.84 | 0.25 to 7 |
| 2 | Mannitol | 296.66 | 50 to 97 |
| 3 | Croscarmellose sodium | 4.5 | 0.5 to 10 |
| 4 | Magnesium stearate | 4.5 | 0.25 to 2 |
| 5 | Colloidal silicon | 1.5 | 0.25 to 2.5 |
| | dioxide | | |
| | Tablet weight (core) | 320.0 | |

Coating

| | | | |
|---|---|---|---|
| 6 | Opadry pink OY 54937 | 12.8 | 2.5 to 5 |
| | Tablet weight (coated) | 332.8 | 100% |

¹actual amounts used
²range of weight % that can be used in a formulation.

Procedure

Same as used in Example 7

### EXAMPLE 9

Coated Tablets containing Amlodipine Maleate 10 mg

| | | Quantity¹ | Quantity² in |
|---|---|---|---|
| S.N. | INGREDIENTS | mg/Tablet | weight % |
| 1 | Amlodipine Maleate | 12.84 | 0.25 to 7 |
| 2 | Pregelatinized starch | 296.66 | 50 to 97 |
| 3 | Crospovidone | 4.5 | 0.5 to 10 |
| 4 | Magnesium stearate | 4.5 | 0.25 to 2 |
| 5 | Colloidal silicon | 1.5 | 0.25 to 2.5 |
| | dioxide | | |
| | Tablet weight (core) | 320.0 | |

Coating

| | | | |
|---|---|---|---|
| 6 | Opadry pink OY | 12.8 | 2.5 to 5 |
| | 0754937 | | |
| | Tablet weight (coated) | 332.8 | 100% |

¹actual amounts used
²range of weight % that can be used in a formulation.
*Opadry OY 0754937 is a mixture of hydroxypropylmethylcellulose (63.65%), titanium dioxide (29.32%), polyethylene glycol 400 (6.3%), Synthetic iron oxide (0.73%).

Procedure

Same as used in Example 7.

### EXAMPLE 10

Uncoated tablets containing Amlodipine Maleate 10 mg

| | | Quantity¹ | Quantity² |
|---|---|---|---|
| S.N. | INGREDIENTS | mg/Tablet | in % |
| 1 | Amlodipine Maleate | 12.84 | 0.25 to 7 |
| 2 | Microcrystalline | 296.66 | 50 to 97 |
| | cellulose | | |
| 3 | Sodium starch | 4.5 | 0.25 to 10 |
| | glycolate | | |
| 4 | Magnesium stearate | 4.5 | 0.25 to 2 |
| 5 | Colloidal silicon | 1.5 | 0.1 to 2.5 |
| | dioxide | | |
| | Tablet weight (core) | 320 | 100% |

¹actual amounts used
²range of weight % that can be used in a formulation.

Amlodipine Maleate Tablets 10 mg
Stability profile (Accelerated stability study)

| Condition of | | | | | % Total |
|---|---|---|---|---|---|
| exposure | Time | | % Impurity 2 | % Impurity 6 | Impurity |
| | Initial | | 0.15 | 0.02 | 0.26 |
| 40° C./75% RH | 3 | Months | 0.14 | 0.30 | 0.74 |
| 25° C./60% RH | 12 | Months | 0.15 | 0.19 | 0.53 |

*Impurities are expressed in percentages by weight.

It was observed that there is not much change in the stability of coated and uncoated tablets of Amlodipine Maleate over a period of 30 days with respect to the level of impurities 2 and 6.

### EXAMPLE 11

Dissolution Profile

Comparative dissolution profile of Amlodipine Maleate vs. Amlodipine Besylate (Norvasc) tablets at various pH:

1. pH 2.1 simulated gastric fluid fasted

Apparatus: USP apparatus II (paddles)

RPM: 50

Volume: 900 ml

| | PRODUCT CONTAINING | % DRUG RELEASE AT DIFFERENT TIME | | | | |
|---|---|---|---|---|---|---|
| | 10 Mg UNCOATED TABLET | INTERVAL | | | | |
| S.N. | DOSAGE FORM | 10 MIN | 20 MIN | 30 MIN | 45 MIN | 60 MIN |
| 1 | AMLODIPINE BESYLATE | 60 | 72 | 80 | 87 | 92 |
| | NORVASC, [REFERENCE] | | | | | |
| 2 | AMLODIPINE MALEATE | 75 | 81 | 84 | 87 | 90 |
| | [TEST] | | | | | |

* All the values are average of 6 readings.

2. pH 4.5 phosphate buffer

Apparatus: USP apparatus II (paddles)

RPM: 50

Volume: 900 ml

| | PRODUCT CONTAINING | % DRUG RELEASE AT DIFFERENT TIME | | | | |
|---|---|---|---|---|---|---|
| | 10 Mg UNCOATED TABLET | INTERVAL | | | | |
| S.N. | DOSAGE FORM | 10 MIN | 20 MIN | 30 MIN | 45 MIN | 60 MIN |
| 1 | AMLODIPINE BESYLATE | 70 | 83 | 90 | 94 | 97 |
| | NORVASC, [REFERENCE] | | | | | |
| 2 | AMLODIPINE MALEATE | 95 | 99 | 100 | 100 | 99 |
| | [TEST] | | | | | |

* All the values are average of 6 readings.

3. pH 7.2 phosphate buffer

Apparatus: USP apparatus II (paddles), RPM: 50, Volume: 900 ml

| | PRODUCT CONTAINING | % DRUG RELEASE AT DIFFERENT TIME | | | | |
|---|---|---|---|---|---|---|
| | 10 Mg UNCOATED TABLET | INTERVALS | | | | |
| S.N. | DOSAGE FORM | 10 MIN | 20 MIN | 30 MIN | 45 MIN | 60 MIN |
| 1 | AMLODIPINE BESYLATE | 60 | 72 | 80 | 87 | 92 |
| | NORVASC, [REFERENCE] | | | | | |
| 2 | AMLODIPINE MALEATE | 75 | 81 | 84 | 87 | 90 |
| | [TEST] | | | | | |

* All the values are average of 6 readings.

Pharmacokinetic Profile

Fasting Studies

Following is the summary of statistical comparisons of the Amlodipine Maleate 10 mg tablets of this invention (Test Fasted) and Norvasc 10 mg tablets (Reference Fasted) when each was administered as a single tablet dose after an overnight fast.

| | Ln-Transformed | | | 90% confidence | |
|---|---|---|---|---|---|
| | Least square means | | | interval | |
| | Test | Reference | Ratio in % | Lower | Upper |
| Parameters | Fasted | Fasted | (Test/Reference) | (%) | (%) |
| AUC₀₋ₜ | 275 | 271 | 101.7 | 98.1 | 105.4 |
| (ng-hr/ml) | | | | | |
| AUC_{0-Inf} | 321 | 314 | 102.3 | 98.2 | 106.6 |
| (ng-hr/ml) | | | | | |
| Cmax | 6.71 | 6.24 | 98.9 | 93.9 | 104.1 |
| (ng-ml) | | | | | |

The data obtained from 36 volunteers. The time needed to reach the maximum blood level concentration was found to be almost identical in case of test and reference products.

Food Study Effect

Following is the summary of statistical comparisons of Amlodipine Maleate 10 mg tablets of this invention when administered as a single tablet dose after a standard high fat breakfast (Test fed) and when administered after overnight fast (Test fasted).

| | Ln-Transformed | | | | |
|---|---|---|---|---|---|
| | Least square means | | | | |
| | Test | Test | Ratio in % | 90% confidence interval | |
| Parameters | Fed | Fasted | (Fed/Fast) | Lower (%) | Upper (%) |
| AUC₀₋ₜ | 284 | 275 | 103.2 | 99.6 | 106.9 |
| (ng-hr/ml) | | | | | |
| AUC_{0-Inf} | 328 | 321 | 102.2 | 98.0 | 106.5 |
| (ng-hr/ml) | | | | | |
| Cmax | 5.85 | 6.17 | 94.8 | 90.1 | 99.7 |
| (ng-ml) | | | | | |

The data obtained from 36 volunteers. The time needed to reach the maximum blood level concentration was found to be almost identical in case of test and reference products during fasting state.

The ratio of AUC & Cₘₐₓ for Test and Reference product was found to be within the accepted limits of FDA as 90% confidence interval fall within 80-125% for log transformed data.

In case of fasting Vs fed studies for test product it is observed that there is no statistical difference in the least square means values of AUC & Cₘₐₓ. This shows that presence of food has no effect on the bioavailability of Amlodipine Maleate tablets.

Thus above statistical pharmacokinetic data suggests that Amlodipine Maleate 10 mg tablets of this invention are bioequivalent to the Norvasc tablets.

Advantages of the Present Invention:

Present invention provides the stable solid orally administrable pharmaceutical formulations comprising Amlodipine Maleate.

The invention also eliminated the common problem of sticking observed while processing Amlodipine Maleate.

The present formulation is proved to be bioequivalent with Amlodipine Besylate formulations commercially available as Norvasc.

The present invention presents the direct compression method to produce stable pharmaceutical formulations of Amlodipine Maleate.

1. A solid orally administrable pharmaceutical formulation comprising Amlodipine Maleate and microcrystalline cellulose, sodium starch glycolate, magnesium stearate and colloidal silicon dioxide.

2. The formulation of claim 1, in the form of a tablet which is optionally coated.

3. The formulation of claim 2, wherein the coating agent is hydroxypropyl methylcellulose.

4. A solid orally administrable pharmaceutical formulation of Amlodipine Maleate comprising:
at least one diluent selected from the group consisting of polyols, sugars, cellulose derivatives and saccharides;
at least one disintegrant selected from the group consisting of clays, gums and polymers;
at least one lubricant selected from the group consisting of Magnesium stearate, stearic acid, glyceryl behenate, behenic acid, calcuim stearate, mineral oil, polyethylene glycol, sodium lauryl sulphate, talc and hydrogenated vegetable oil; and
at least one glidant selected from the group consisting of colloidal silicon dioxide and talc.

5. The formulation of claim 4, wherein polyol is selected from the group consisting of mannitol, sorbitol and xylitol.

6. The formulation of claim 4, wherein the sugar is selected from the group consisting of sucrose, lactose and dextrose.

7. The formulation of claim 4, wherein the saccharide is selected from the group consisting of dextrates, maltodextrans, starch, pregelatinized starch and cyclodextrin.

8. The formulation of claim 4, wherein the cellulose derivative is selected from the group consisting of microcrystalline cellulose and powdered cellulose.

9. The formulation of claim 4, wherein the clay is selected from the group consisting of bentonite and vegum.

10. The formulation of claim 4 wherein the gum is selected from the group consisting of sodium alginate, xanthan gum, veegam and guar gum.

11. The formulation of claim 4 wherein the polymer is selected from the group consisting of sodium starch glycolate, crosscarmellose sodium, crosslinked polyvinylpyrollidone and crospovidone.

12. The formulation according to any one of claims 1 to 11, in the form of tablet, caplet or capsule.

13. The formulation of claim 12, wherein the tablet is coated.

14. The formulation of claim l,comprising an antihypertensive or antianginal alleviating effective amount of Amlodipine Maleate.

15. The formulation of claim 4 comprising an antihypertensive or antianginal alleviating effective amount of Amlodipine Maleate.

16. A formulation comprising about 0.25 to about 7% by weight of amlodipine maleate; about 50 to about 97% by weight of a diluent; about 0.5 to about 10% by weight of a disintegrant; about 0.25 to about 2% by weight of a lubricant and about 0.1 to about 2.5% by weight of a glidant.

17. The formulation according to claim 16 wherein the diluent is microcrystalline cellulose, the disintegrant is sodium starch glycolate, the lubricant is magnesium stearate and the glidant is colloidal silicon dioxide.

18. The formulation according to claim 16 wherein the diluent is mannitol, the disintegrant is croscarmellose sodium, the lubricant is magnesium stearate and the glidant is colloidal silicon dioxide.

19. The formulation according to claim 16 wherein the diluent is pregelatinized starch, the disintegrant is crospovidone, the lubricant is magnesium stearate and the glidant is colloidal silicon dioxide.

20. A formulation comprising about 0.25 to about 7% by weight of amlodipine maleate, about 50 to about 97% by weight of microcrystalline cellulose, about 0.5 to about 10% by weight of sodium starch glycolate, about 0.25 to about 2% by weight of magnesium stearate; and about 0.1% to about 2.5% by weight of colloidal silicon dioxide.

21. A formulation comprising about 1 to about 5% by weight of amlodipine maleate, about 75 to about 95% by weight of microcrystalline cellulose, about 0.5 to about 5% by weight of sodium starch glycolate, about 0.5 to about 2% by weight of magnesium stearate; and about 0.2 to about 2% by weight of colloidal silicon dioxide.

22. A method for preparing a formulation according to claim 4 comprising the steps of:
a) blending amlodipine maleate, the diluent and the disintegrant for about.

37. A method for treating hypertension comprising administering an effective amount of a formulation of claim 20 to a patient in need thereof.

38. A method for treating hypertension comprising administering an effective amount of a formulation of claim 21 to a patient in need thereof.
